(19) United States
(12) Patent Application Publication
(10) Pub. No.: US 2016/0178264 A1
(43) Pub. Date: Jun. 23, 2016

(54) Temperature gradient system and method

(71) Applicant: The Trustees of Dartmouth College, Hanover, NH (US)

(72) Inventors: Rachel W. Obbard, Hartland, VT (US); Natalie P. Afonina, Mill Creek, WA (US); Ross Lieb-Lappen, Norwich, VT (US); Charles Gunnar Pope, Hanover, NH (US)

(21) Appl. No.: 14/757,648

(22) Filed: Dec. 23, 2015

Related U.S. Application Data
(60) Provisional application No. 62/096,037, filed on Dec. 23, 2014.

(51) Int. Cl. F25D 11/00; F25D 11/02; A61F 7/00; F25D 29/00; B65B 5/04; B65B 63/08; F25B 21/04; F25D 23/00

(52) U.S. Cl. CPC F25D 11/003; F25B 21/04; F25D 11/02; F25D 23/00; F25D 29/003; B65B 5/04; B65B 63/08; A61F 7/007; A61F 2007/0002; A61F 2007/0018; A61F 2007/0029; A61F 2007/0039; A61F 2007/0075; A61F 2007/0086; A61F 2007/0096

(57) ABSTRACT

A temperature gradient system includes an insulated container and heat pumps. Temperature sensing devices measure temperature and a controller controls heat pumps to different temperatures at different positions of the insulated container to produce a temperature gradient. A temperature gradient method includes insulating a sample and controlling temperature to maintain a temperature gradient lengthwise along the sample. The sample is an ice core, in a particular embodiment, and different temperatures are maintained at opposite ends of the insulated container. The sample is a portion of a body, in another embodiment, and the system is configured to accommodate a particular portion of the body for controlled cooling and heating.

### BACKGROUND

In nature, the temperature of sea ice is approximately −2° C. at the ice-water interface (bottom) and approximately surface air temperature, for example −35° C. at the surface. Prior art systems and methods for storing ice cores maintain a single temperature. Because sea ice has complex chemistry and microstructure that is temperature dependent, systems and methods that impose a single temperature irreversibly change its microstructure, adversely affecting its study.

In medicine, targeted temperature management includes lowering body temperature to reduce deleterious effects of low blood flow caused by cardiac arrest, stroke, or traumatic brain injury. Different temperatures may be targeted for different portions of a body such as limbs, and normal body temperature is returned in a controlled manner.

### SUMMARY OF THE INVENTION

According to an embodiment, a temperature gradient system is provided. The system an insulated container; a first heat pump located at a first end of the insulated container; and a second heat pump located at a second end of the insulated container, wherein the second end is opposite the first end. The system further includes a controller for controlling the first heat pump to maintain a first temperature and the second heat pump to maintain a second temperature, wherein the first and second temperatures are different, thereby maintaining a temperature gradient between ends of the insulated container.

According to another embodiment, a temperature gradient method is provided. The method includes wrapping a sample with a low emissivity insulating layer, placing the wrapped sample inside an insulated container, and measuring temperature at a first end and a second end of the insulated container. The method further includes controlling a first heat pump located at a first end of the insulated container to provide a first temperature and a second heat pump located at a second end to provide a second temperature; maintaining a temperature gradient where the first and second temperatures are different; and removing heat from first and second ends with a first heat pump and a second heat pump, respectively.

According to yet another embodiment, a temperature gradient system is provided. The system includes an insulated container having a compartment configured to accommodate a portion of a body; a plurality of thermal zones for controlling temperature of the compartment; at least one heat pump thermally connected to the plurality of thermal zones, wherein the at least one heat pump includes one or more thermoelectric modules; and a controller for controlling the one or more thermoelectric modules to maintain a desired temperature in the thermal zones for controlling temperature to the portion of the body.

### BRIEF DESCRIPTION OF THE FIGURES

FIG. 1 is a diagram showing one embodiment of a temperature gradient system.

FIG. 2 is a cross-sectional view lengthwise through the center of one embodiment of a temperature gradient system.

FIG. 3 shows one embodiment of a temperature gradient method.

FIG. 4 is a schematic drawing showing one embodiment of a heat pump and thermal conductor used in a temperature gradient system.

FIG. 5 is a diagram showing one embodiment of a heat pump for a temperature gradient system.

FIG. 6 is a cross-sectional view lengthwise through the center of one embodiment of a temperature gradient system with two compartments.

FIG. 7 is a schematic drawing showing one embodiment of a heat pump and two thermal conductors used in a temperature gradient system.

FIG. 8 is a diagram illustrating exemplary details of a controller for a temperature gradient system.

FIG. 9 is a cross-sectional view lengthwise through the center of another embodiment of a temperature gradient system.

FIG. 10 shows one embodiment of a temperature gradient system for legs.

FIG. 11 shows one embodiment of a temperature gradient system for an arm.

FIG. 12 shows one embodiment of a temperature gradient system for a torso and a head.

### DETAILED DESCRIPTION OF THE EMBODIMENTS

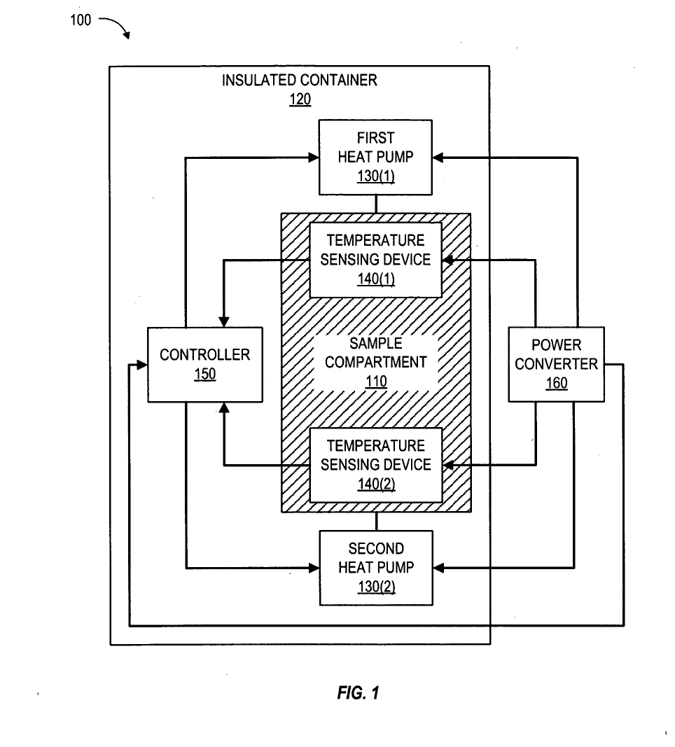

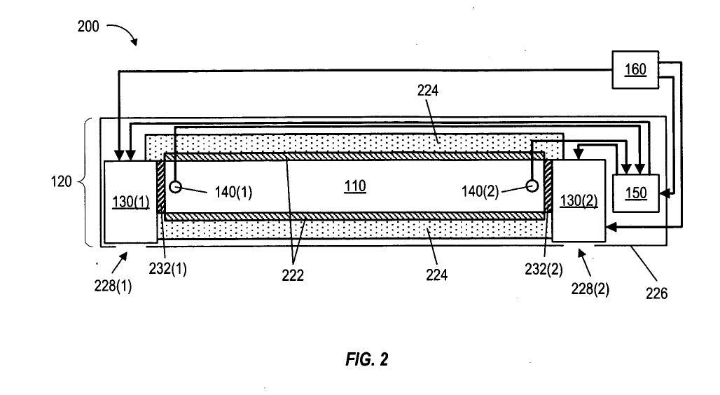

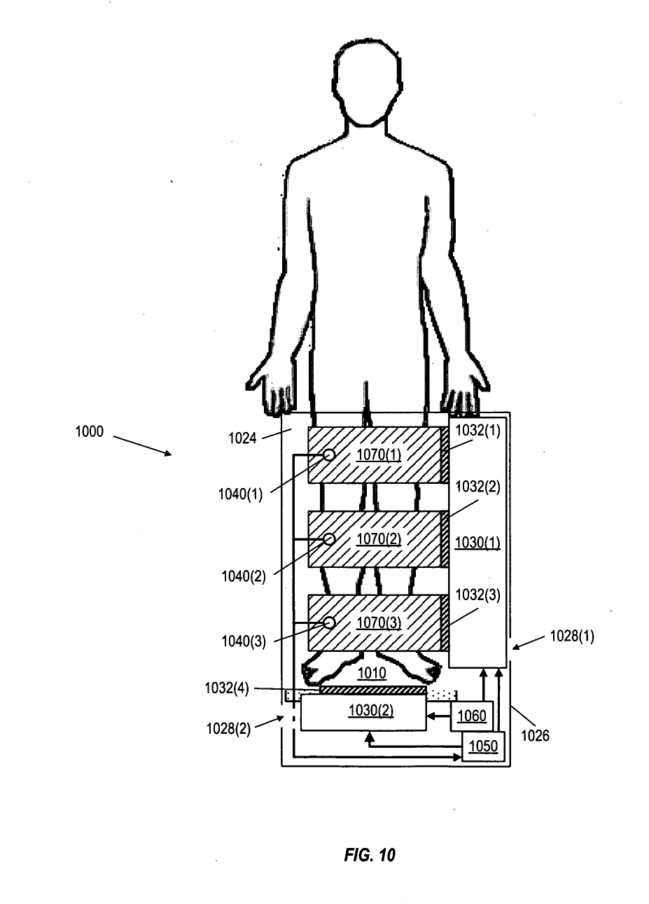

FIG. 1 is a diagram showing one embodiment of a temperature gradient system 100. System 100 provides a temperature gradient to a sample compartment 110. In some embodiments, sample compartment 110 is configured to accommodate ice core samples (FIGS. 2, 6, and 9). Example ice core samples include cores of ice from sea ice, lake ice, pond ice, river ice, or glacier ice, although non-ice samples are also possible. System 100 provides a temperature gradient along the ice core that approximates a natural temperature gradient of the ice from which the core was retrieved in order to preserve its microstructure. In other embodiments, sample compartment 110 is configured to accommodate portions of an animal or human body (FIGS. 10-12).

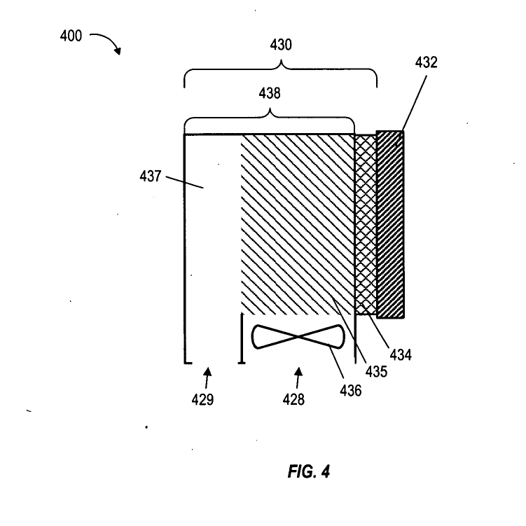

In FIG. 1, sample compartment 110 is located inside an insulated container 120 that provides thermal insulation and protection for ice cores during transport. In an embodiment, container 120 is a box or container suitable for holding, transporting, and insulating one or more ice cores. A first heat pump 130(1) is located at a first end of insulated container 120 for thermally connecting to a first end of compartment 110. A second heat pump 130(2) is located at a second opposite end of container 120 for thermally connecting to a second end of compartment 110. Heat pumps 130(1), 130(2) are for example heat sinks, solid state heat pumps, liquid heat pumps, air heat exchangers, liquid heat exchangers, refrigerators, or a combination of two or more of these. See FIGS. 4, 5, and 7 for exemplary heat pumps.

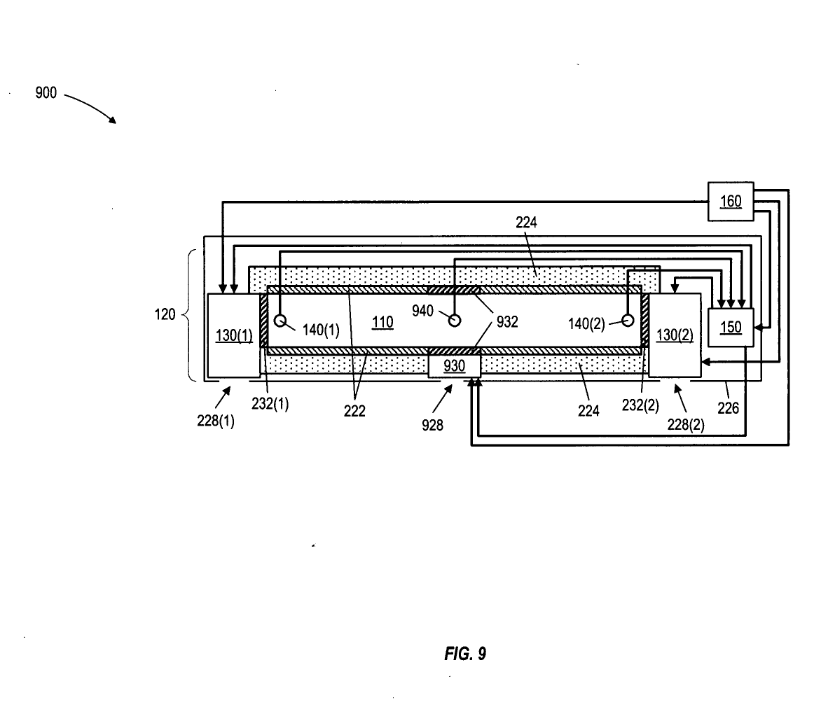

A first temperature sensing device 140(1) and a second temperature sensing device 140(2) are placed inside sample compartment 110 near first and second ends, respectively, for measuring sample temperature. Temperature sensing devices 140(1), 140(2) are for example thermocouples, thermistors, or resistance temperature detectors, which are electronically coupled to a controller 150 for providing temperature information. In an embodiment, temperature sensing devices 140(1), 140(2) are placed inside holes drilled into the sample near each end. Placement of temperature sensing devices 140(1), 140(2) inside the sample provides increased accuracy but requires drilling into the sample. Alternatively, temperature sensing devices 140(1), 140(2) are located inside compartment 110 adjacent to the sample, at the ends of compartment 110. A third temperature sensing device may be placed near the middle of the sample (see FIG. 9) and additional temperature sensing devices may be placed elsewhere without departing from the scope hereof. For example, temperature sensing devices may be placed every 10 cm along the length of sample compartment 110.

System 100 includes a power converter 160 to provide electrical power to all components requiring it, including controller 150, heat pumps 130(1), 130(2), and if necessary, temperature sensing devices 140(1), 140(2). Power converter 160 includes an AC/DC converter that converts 120V alternating current (AC) to direct current (DC) at 18V or 12V for example, enabling system 100 to be plugged into a standard electrical socket. Power converter 160 is shown outside container 120 in FIG. 1 but may be located inside container 120. Electrical power may be provided by a generator, batteries, or another suitable source of electricity.

Controller 150 is electrically connected to first and second heat pumps 130(1), 130(2) and first and second temperature sensing devices 140(1), 140(2). Controller 150 uses temperature information, received from temperature sensing devices 140(1), 140(2), to control first heat pump 130(1) to maintain a first temperature and second heat pump 130(2) to maintain a second temperature. By setting first and second temperatures to be different, a temperature gradient is maintained through the length of sample compartment 110. For example, system 100 forms a temperature gradient by maintaining a temperature difference of 38° C. between first and second ends of sample compartment 110 for ambient temperatures between −35° C. and 25° C.

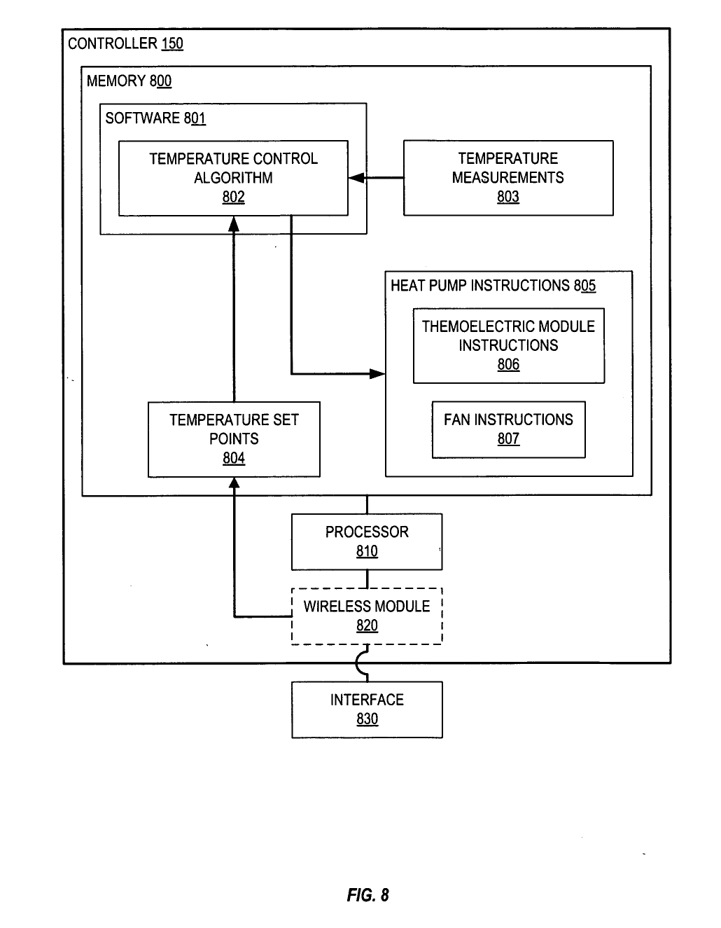

First and second temperatures are set by user inputs to controller 150. Controller 150 compares user defined temperature set points with measured temperatures from temperature sensing devices 140(1), 140(2), then appropriately adjusts electrical current to heat pumps 130 to minimize differences between the set points and the measured temperatures. Controller 150 includes a temperature control algorithm, such as a proportional-integral-derivative (PID) control feedback loop for example. FIG. 8 shows controller 150 in exemplary detail.

FIG. 2 is a cross-sectional view lengthwise through the center of one embodiment of a temperature gradient system 200. System 200 is an example of system 100 of FIG. 1. System 200 includes components of system 100, specifically sample compartment 110, insulated container 120, first and second heat pumps 130(1), 130(2), first and second temperature sensing devices 140(1), 140(2), controller 150, and power converter 160. System 200 further includes a first thermal conductor 232(1) located between first heat pump 130(1) and first end of sample compartment 110, and a second thermal conductor 232(2) located between second heat pump 130(2) and second end of sample compartment 110. First and second thermal conductors 232(1), 232(2) make physical contact between their respective heat pump 130(1), 130(2) and the respective end of the sample in compartment 110 to provide conduction for efficient heat transfer. Thermal conductors 232(1), 232(2) are plates made of metal such as aluminum or copper for example. In an embodiment, first and second thermal conductors 232(1), 232(2) are removable from system 200. In an event of power loss, thermal conductors 232(1), 232(2) are removed and optionally replaced with insulation to advantageously disrupt heat transfer. Without power, system 200 in a particular embodiment maintains sample temperatures within 3° C. and a gradient of 35° C. for up to two hours. This allows an ice core sample to be transported by air for short flights without power.

System 200 also includes a low emissivity wrapping 222 around the sample. The length of low emissivity wrapping 222 is matched to the length of the sample but does not cover the sample ends, which contact thermal conductors 232(1), 232(2). In an embodiment, low emissivity wrapping 222 provides an inner insulation layer of insulated container 120 and is made of material that reflects radiation thereby reducing radiative heat transfer. Insulated container 120 further includes middle insulating layer 224 that surrounds low emissivity wrapping 222. Middle insulating layer 224 is made for example of fiberglass insulation and fills gaps inside insulated container 120. In an embodiment, middle insulating layer 224 includes polystyrene foam. An outer cover 226 surrounding middle insulating layer 224 provides an additional layer of insulation and structural support for components of system 200. Outer cover 226 has for example a substantially sealed enclosure thereby preventing external airflow for maintaining a heat transfer barrier. In an embodiment, insulated container 120 of FIG. 1 includes low emissivity wrapping 222, middle insulating layer 224, and outer cover 226, which oppose heat transfer. Heat transfer is opposed in the longitudinal direction, except within the sample itself, to maintain a temperature gradient between first and second ends. Additionally, heat transfer is opposed in the radial direction to thermally isolate the sample from ambient air.

Outer cover 226 includes a first thermal vent 228(1) located adjacent to first heat pump 130(1) and a second thermal vent 228(2) located adjacent to second heat pump 130(2). First and second thermal vents 228(1), 228(2) are for example holes cut through outer cover 226 to enable heat transfer from first and second heat pumps 130(1), 130(2) to outside system 200. First and second thermal vents 228(1), 228(2) have for example screens or louvers that cover the holes of outer cover 226, thus protecting heat pumps 130(1), 130(2) while enabling heat transfer. Outer cover 226 includes handles for transporting and a lid for accessing sample compartment 110. Outer cover 226 also includes double overlapped joints, on its lid for example, to reduce external air exchange. Outer cover 226 provides structural support that enables transporting samples without damage, including transport by sled or truck over rugged terrain. In an embodiment, outer cover 226 includes a rigid polystyrene foam bed shaped to fit samples wrapped with low emissivity layer 222 and to fit middle insulating layer 224.

Heat pump 130(1) and thermal conductor 232(1) at first end of sample compartment 110 are moveable to accommodate a sample of shorter length. In an embodiment, heat pump 130(1) and thermal conductor 232(1) are mountable at more than one location in insulated container 120 enabling multiple potential positions. In an embodiment, heat pump 130(1) and thermal conductor 232(1) are mounted on rails for sliding to multiple positions along the length of sample compartment 110. When heat pump 130(1) and thermal conductor 232(1) are repositioned to accommodate a shorter sample, vent 228(1) is for example connected to heat pump 130(1) with a flexible tube to maintain a path for air exchange.

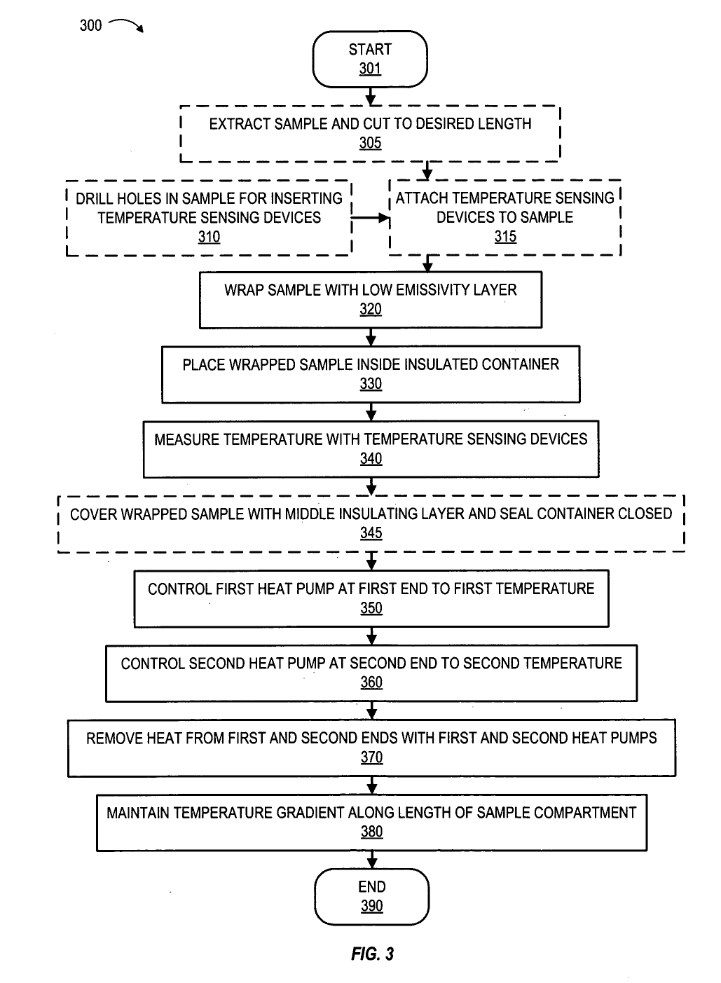

FIG. 3 shows one embodiment of a temperature gradient method 300. FIG. 3 is best viewed together with FIGS. 1 and 2. After starting 301, optional step 305 extracts the sample and cuts it to a desired length. In an embodiment, the sample is a cylindrical core of ice, 14 cm in diameter and 1 m long. Optional step 310 drills holes into the sample for inserting temperature sensing devices at different positions along the sample length, including near both ends, and possibly at additional locations such as in the middle or every 10 cm. Optional step 315 attaches temperature sensing devices 140(1), 140(2) to the sample and into drilled holes of the sample if available. Step 320 wraps the sample with low emissivity insulating layer 222. Step 330 places the wrapped sample inside compartment 110 of insulated container 120. Step 340 measures temperature with temperature sensing devices 140(1), 140(2) near the first and second ends of sample compartment 110. Additional temperature sensing devices are optionally used to measure temperature at additional locations along the length of the sample, such as in the middle or every 10 cm, enabling gradient monitoring. Optional step 345 covers the wrapped sample with middle insulating layer and seals container 120 closed. In an embodiment, step 345 seals container 120 with outer cover 226, which includes double overlapped joints on its lid preventing external airflow.

Step 350 controls first heat pump 130(1) located at first end of sample compartment 110 to a first temperature using controller 150. Step 360 controls second heat pump 130(2) located at second end of sample compartment 110 to a second temperature using controller 150. In an embodiment, first temperature differs from second temperature to provide a temperature gradient along the length of sample compartment 110. Step 370 removes heat from first and second ends of sample compartment 110 using first and second heat pumps 130(1), 130(2), respectively. Step 380 maintains a temperature gradient along the length of insulated container 120 by controlling first and second temperatures with controller 150. In an embodiment, step 380 maintains a temperature difference of 38° C. between first and second ends of sample compartment 110 at ambient temperatures between −35° C. and 25° C. Step 390 ends method 300.

FIG. 4 is a schematic drawing 400 showing one embodiment of a heat pump 430 and thermal conductor 432 used in a temperature gradient system, such as system 100 of FIG. 1 or system 200 of FIG. 2. Heat pump 430 is an example of first and second heat pumps 130(1), 130(2) of FIGS. 1 and 2. Thermal conductor 432, which is an example of first and second thermal conductors 232(1), 232(2) of FIG. 2, makes physical contact with heat pump 430 for conductive heat transfer with sample compartment 110. Located adjacent to, and in thermal contact with, thermal conductor 432, is a thermoelectric module 434. Thermoelectric module 434 is a solid-state thermoelectric device such as a thermoelectric cooler or Peltier-effect device. Such devices transfer heat from one side to another side when a direct electrical current is applied. Direction of heat transfer depends on the direction of applied current. Thermoelectric module 434 is configured to transfer heat from a first side, adjacent to thermal conductor 432, towards a second side opposite the first side. In an embodiment, thermoelectric module 434 is a series of cascaded thermoelectric modules, also known as a multistage thermoelectric module.

Located in physical contact with the second side of thermoelectric module 434 is heat sink 435. Heat sink 435 is for example a plurality of thermally conductive fins that provide a large surface area for convective cooling to dissipate heat to air. In an embodiment, heat sink 435 is made from metal, such as aluminum, by extruding material to create fins and anodizing the surface. Heat sink 435 removes heat from the hot side of thermoelectric module 434 and dissipates it to ambient air. This may occur passively by natural convection or actively using forced convection. Forced convection is for example provided by a fan 436.

Fan 436 is configured to move air across fins of heat sink 435 to outside system 200 for example. Fan 436 exchanges hot air from heat sink 435 with cooler ambient air to actively dissipate heat. Air flow through fan 436 may proceed in either direction to pull cooler air in or push hot air out. A first, vent 428 and a second vent 429 serve as either inlet or outlet for air flow depending on the direction of flow from fan 436. Vents 428, 429 are an example of thermal vent 228 of FIG. 2. In an embodiment, vent 429 is located on the far side of heat sink 435 opposite fan 436, as depicted in FIG. 2. A duct 437 provides a path for air to circulate. When fan 436 is configured to pull ambient air through vent 428, air is pushed through fins of heat sink 435, and hot air returns outside through duct 437 and vent 429. When fan 436 is configured to push hot air out vent 428, ambient air is pulled in via vent 429, through duct 437, and through fins of heat sink 435. In an embodiment, heat sink 435, fan 436, duct 437, and vents 428, 429 together form a heat exchanger 438. Heat exchanger 438, which typically exchanges heat with air, may be configured to exchange heat with a liquid such as sea water.

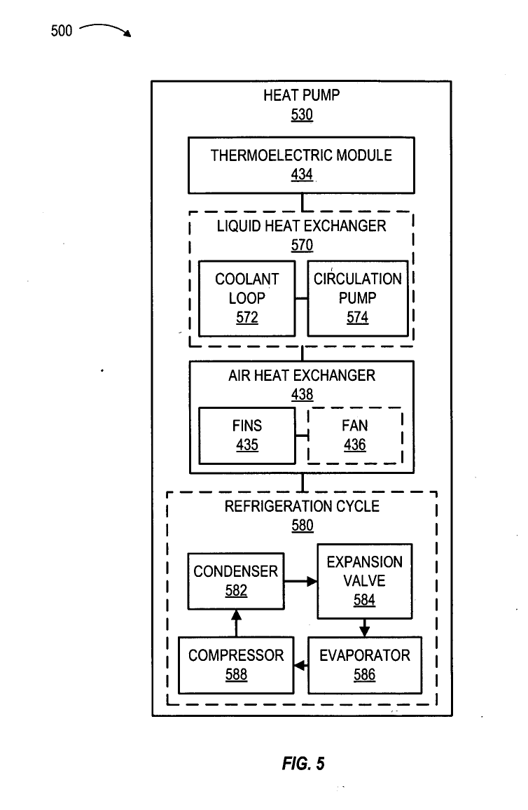

FIG. 5 is a diagram showing one embodiment of a heat pump 530 for a temperature gradient system. Heat pump 530 is an alternative example of heat pump 130 of FIG. 1. Heat pump 530 includes thermoelectric module 434 and heat exchanger 438 of FIG. 4. In addition, heat pump 530 includes optional liquid heat exchanger 570 and optional refrigeration cycle 580. Liquid heat exchanger 570 includes a coolant loop 572 and a circulation pump 574. Circulation pump 574 is a peristaltic pump for example configured to circulate coolant through coolant loop 572. Coolant loop 572 provides a path to circulate a liquid between one or more thermoelectric modules 434 and heat exchanger 438. Alternatively, coolant loop 572 provides a thermal connection between distantly located thermal conductor 432 and thermoelectric module 434. Coolant loop 572 is for example a flexible polyethylene tube. In an embodiment, coolant loop 572 includes a TYGOTHANE™ C-210-A tube. Example low freezing point coolants that may be contained within coolant loop 572 include alcohol, ethylene glycol anti-freeze, propylene glycol, and solutions containing mixtures of one or more of these liquids with water. In an embodiment, liquid heat exchanger 570 replaces thermoelectric module 434, which may be achieved by configuring coolant loops 572 to make thermal contact with thermal conductors 432 and using digital-proportional valves to control coolant flow rate via controller 150.

Optional liquid heat exchanger 570 provides potential advantages. For example, because heat exchangers need to be large in order to rapidly dissipate large amounts of heat, having fewer heat exchangers or locating them outside outer cover 226 may be advantageous. Coolant loop 572 is configured to circulate liquid to a plurality of thermoelectric modules and transfer heat to fewer air heat exchangers, which may be located outside outer cover 226. Locating heat exchanger 438 outside cover 226 enables exchange of heat with sea water for example. Another advantage is to enable thermal conductor 232(1), FIG. 2 to be moveable to accommodate shorter samples. In an embodiment, coolant loop 572 circulates inside thermal conductors 232(1), 232(2), FIGS. 2 and 432, FIG. 4 and is extendable for maintaining thermal contact between distantly located heat pump 130(1) and sample compartment 110. In an embodiment, coolant loops are copper coils sandwiched between aluminum plates that make up thermal conductors 232(1), 232(2) and 432.

Similarly, refrigeration cycle 580 uses a liquid coolant that is configured to cool multiple thermoelectric modules 434 or distantly located thermal conductors 432. Refrigeration cycle 580 includes condenser 582, which transfers heat to heat exchanger 438, and evaporator 586, which absorbs heat from thermoelectric module 435. Refrigeration cycle provides a heat transfer advantage over liquid heat exchanger 570 because it effectively increases thermal capacity of coolant by taking advantage of latent heat during coolant phase transitions. This is achieved by rapidly decreasing coolant pressure with expansion valve 584, configured to bring coolant from a cool liquid to a liquid/vapor mixture, and by rapidly increasing pressure with compressor 588, configured to bring coolant from a vapor to a hot liquid.

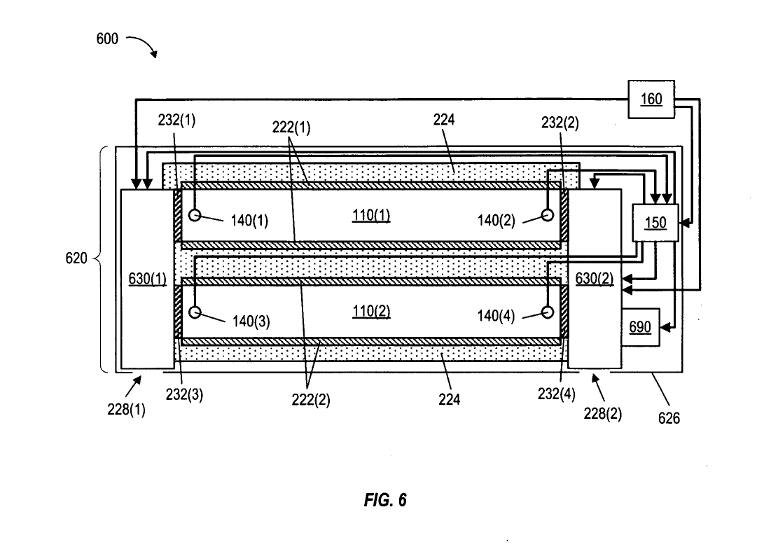

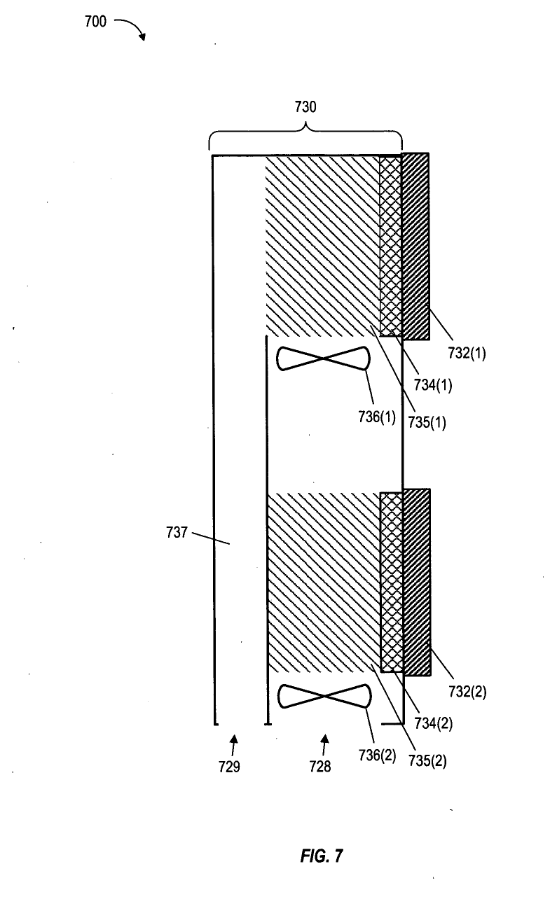

FIG. 6 is a cross-sectional view lengthwise through the center of one embodiment of a temperature gradient system 600. System 600 is an example of system 200 of FIG. 2 and includes insulated container 620, which is an example of insulated container 120 configured with a first sample compartment 110(1) and a second sample compartment 110(2). FIG. 6 shows two sample compartments 110(1), 110(2), but system 600 may be configured with more than two compartments for more than two samples without departing from the scope hereof. System 600 includes at least two heat pumps, one for each end of container 620. In an embodiment, a first heat pump 630(1) and a second heat pump 630(2) are configured to control temperature at a first and second end of sample compartments 110(1), 110(2), respectively. Heat pumps 630(1), 630(2) are examples of heat pumps 130(1), 130(2) of FIG. 2. In an alternative embodiment, four heat pumps 630 are provided, one for each end of each compartment 110(1), 110(2). Heat pumps 630(1), 630(2) of FIG. 6 each have two thermoelectric modules 434 (see FIG. 7) for independently controlling temperature at one end of each sample 110(1), 110(2), respectively. Heat pumps 630(1), 630(2) have one or more heat exchangers 438 of FIG. 4 in order to remove heat from one end of two samples 110(1), 110(2) through vents 228(1), 228(2), respectively. In an embodiment, vents 228(1), 228(2) each have an inlet and outlet vent, such as vents 428, 429 of FIG. 4. Heat pumps 630(1), 630(2) include a duct configured to provide a path for exchange of hot air from one or more heat exchangers with ambient air, similar to duct 437 of FIG. 4 for example.

First heat pump 630(1) includes a first and second thermoelectric module 434 (see FIG. 7) for providing a first and second temperature, respectively. Similarly, second heat pump 630(2) includes a third and fourth thermoelectric module 434 for providing a third and fourth temperature, respectively. First and third temperatures are different for maintaining a temperature gradient in first sample compartment 110(1). Similarly, second and fourth temperatures are different for maintaining a temperature gradient in second sample compartment 110(2). In an embodiment, system 600 includes at least one additional heat pump between the ends of sample compartments 110(1), 110(2) to supplement heat pumps 630(1), 630(2). See FIG. 9 and accompanying description below.

Third and fourth temperature sensing devices 140(3), 140(4) are examples of first and second temperature sensing devices 140(1), 140(2) located near first and second ends, respectively, of second sample compartment 110(2). System 600 includes controller 150 configured to control first and second compartments 110(1), 110(2) for providing independent temperature gradients. Temperature sensing devices 140(1-4) provide temperature information to controller 150 and receive power from power converter 160 if necessary. Connections between temperature sensing devices 140(1-4) and power converter 160 are not shown in FIG. 6 for clarity of illustration. System 600 includes low emissivity wrapping 222(1), 222(2) around samples in first and second compartments 110(1), 110(2), respectively. Middle insulating layer 224 surrounds and separates both sample compartments 110(1), 110(2). An outer cover 626 surrounding middle insulating layer 224 provides an additional layer of insulation and structural support for components of system 600. Outer cover 626 is an example of outer cover 226 of FIG. 2 configured for two sample compartments 110(1), 110(2) instead of one. A third thermal conductor 232(3) is located between first heat pump 630(1) and first end of second sample compartment 110(2), and a fourth thermal conductor 232(4) is located between second heat pump 630(2) and second end of second sample compartment 110(2). Third and fourth thermal conductors 232(2), 232(4) make physical contact between their respective heat pump and sample end to provide conduction for efficient heat transfer. In an embodiment, thermal conductors 232(3), 232(4) are removable to reduce heat transfer when system 600 is without power.

To accommodate samples of shorter length, thermal conductors 232(1), 232(3) at first ends of sample compartments 110(1), 110(2) are moveable. In an embodiment, thermal conductors 232(1), 232(3) are independently mountable at more than one location inside sample compartments 110(1), 110(2), respectively, enabling multiple potential positions. Or for example, thermal conductors 232(1), 232(3) are mounted on rails inside sample compartments 110(1), 110(2) for independently sliding to multiple positions for accommodating various length samples. In an alternative embodiment, thermal conductors 232(1), 232(3) are clamped to first ends of samples with adjustable clamps. Thermal conductors 232(1), 232(3) remain thermally connected to heat pump 630(1) despite distance between them by a coolant loop, such as coolant loop 572, FIG. 5 for example.

System-600 includes power amplifier 690 located in physical contact with heat pump 630(2). Power amplifier 690 is controlled by controller 150 to provide high electrical current to thermoelectric modules 434 for rapid cooling of sample compartments 110. Controller 150 supplies for example a pulse-width modulated voltage to power amplifier 690. In an embodiment, power amplifier 690 is located on a printed circuit board. Power amplifier 690 generates heat that is transferred to heat pump 630(2) by conduction and removed from system 600 by heat exchanger 438.

FIG. 7 is a schematic drawing 700 showing one embodiment of a heat pump 730 and two thermal conductors 732(1), 732(2) used in a temperature gradient system, such as system 600 of FIG. 6. Thermal conductors 732(1), 732(2) are examples of first and third thermal conductors 232(1), 232(3) of FIG. 6. Heat pump 730 is an example of heat pump 430 of FIG. 4 that includes two thermoelectric modules 734(1), 734(2), two heat sinks 735(1), 735(2) , and two fans 736(1), 736(2), which are examples of thermoelectric module 434, heat sink 435, and fan 436 of FIG. 4, respectively. In an embodiment, heat pump 730 is configured with only one fan 736 without departing from the scope hereof. Duct 737 and vents 728, 729 are examples of duct 437 and vents 428, 429 of FIG. 4, respectively. Ambient air passes through both heat sinks 735(1), 735(2) to remove heat from thermoelectric modules 734(1), 734(2). In an embodiment, heat pump 730 is configured with only one larger heat sink 735 adjacent to both thermoelectric modules 734(1), 734(2) and one fan 736 for air heat exchange. Thermoelectric modules 734(1), 734(2) are independently controlled by controller 150 for providing independent temperatures.

FIG. 8 is a diagram illustrating exemplary details of controller 150. Controller 150 is for example a digital computer, programmable controller, programmable logic controller, or programmable logic relay. In an embodiment, controller 150 is an Arduino Mega 2560. Controller 150 includes for example non-volatile memory 800, software 801, a processor 810, and an interface 830. Memory 800 stores software 801 that includes machine readable instructions that when executed by processor 810 provide control and functionality of system 100 as described herein. Software 801 includes code that defines a temperature control algorithm 802, such as a proportional-integral-derivative (PID) control feedback loop for example.

Processor 810 executes software 801, which uses temperature control algorithm 802, temperature measurements 803, and user defined temperature set points 804, and provides instructions to control heat pumps 805. Heat pump instructions 805 include for example thermoelectric module instructions 806 and fan instructions 807. Thermoelectric module instructions 806 provide instructions for controlling electrical current provided to thermoelectric module 434 of FIG. 4 for example. In an embodiment, thermoelectric module instructions 806 provide instructions for independently controlling electrical current provided to thermoelectric modules 734(1), 734(2) of FIG. 7. Fan instructions 807 provide instructions for controlling fan 436 of FIG. 4 or fans 736(1), 736(2) of FIG. 7 for example. Fan 436 may be a single speed fan, in which case fan instructions 807 may cycle power to fan 807 on and off depending on the thermal load produced by heat sink 435. In an embodiment, fan 436 is a variable speed fan, in which case fan instructions 807 control the speed of fan 436 depending on thermal load produced by heat sink 435. The thermal load of heat sink 435 may be determined from a dedicated temperature sensing device 140 or calculated based on power supplied to thermoelectric module 434.

Controller 150 has for example electronic circuitry including relays and switches to electrically connect with system 100 components including temperature sensing device 140, heat pump 130, power converter 160, power amplifier 690 of FIG. 6, and optional wireless module 820. In an embodiment, wireless module 820 is an Xbee Series 2 transceiver that provides radio communication between controller 150 and an interface 830. Interface 830 is for example a computer with capability to connect wirelessly through a wireless modem. Temperature measurements 803 are received from temperature sensing devices 140(1-4) by controller 150 and passed to interface 830 via wireless module 820 for example. Interface 830 accepts user-defined inputs such as temperature set points 804 and modifications to temperature control algorithm 802 for example.

FIG. 9 is a cross-sectional view lengthwise through the center of an exemplary temperature gradient system 900. System 900 is an example of system 200, FIG. 2 and includes many of the same components of system 200. Accordingly, description of components illustrated with like numerals will not be repeated here. System 900 includes a third heat pump 930, which is an example of first and second heat pumps 130(1), 130(2) of FIGS. 1 and 2. System 900 includes three heat pump 130(1), 130(2), 930 but may be configured with more than three heat pumps without departing from the scope hereof. System 900 further includes a third temperature sensing device 940, a third thermal conductor 932, and a third heat thermal vent 928, which are examples of first temperature sensing device 140(1), first thermal conductor 232(1), and first thermal vent 228(1), FIG. 2. Third heat pump 930 is located in about the middle of sample compartment 110 and includes means to maintain a third desired temperature, such as thermoelectric module 434, FIG. 4, liquid heat exchanger 570, FIG. 5 or refrigeration cycle 580, FIG. 5. The third temperature may be the same or different than first and second temperatures at each end of sample compartment 110. According to one embodiment, the third temperature is the average of the first and second temperatures. According to another embodiment, third heat pump 930(3) and optionally additional heat pumps are used to provide a non-uniform temperature gradient.

FIGS. 10, 11, 12 show exemplary temperature gradient systems 1000, 1100, 1200 configured for temperature control of portions of a body. Systems 1000, 1100, 1200 each include one or more compartments configured to accommodate different portions of the body, as depicted in FIGS. 10, 11, 12, respectively. Controlling body temperature is often used as a medical procedure referred to as targeted temperature management. Targeted temperature management typically reduces body temperature of a patient to reduce the risk of tissue injury from inadequate blood flow caused by a medical condition such as cardiac arrest, stroke, or traumatic brain injury. Systems 1000, 1100, and 1200 may be used to maintain a reduced body temperature and to return to normal body temperature in a controlled manner. In particular, systems 1000, 1100, and 1200 may be used to provide a temperature gradient along a portion of the body. For example, limbs are typically chilled by a greater amount relative to core body temperature, and the hands and feet may be chilled by the greatest amount requiring a gradient along the limbs. Systems 1000, 1100, and 1200 may be used in one or more combinations together to manage overall body temperature and these systems may be configured for use with animals during veterinary procedures.

FIG. 10 shows an exemplary temperature gradient system 1000 configured for temperature control of a user's legs. System 1000 is an example of system 100 of FIG. 1 and system 900 of FIG. 9 that provides multi-zone temperature control of a patient's legs. System 1000 includes a compartment specifically configured as a leg compartment 1010, a controller 1050, and a power converter 1060, which are examples of sample compartment 110, controller 150, and power converter 160, FIG. 1. An outer cover 1026 and an insulating layer 1024 are examples of outer cover 226 and middle insulating layer 224, FIG. 2. Insulating layer 1024 optionally includes a low emissivity wrapping applied around the legs, similar to low emissivity wrapping 222, FIG. 2, which is not shown in FIG. 10 for clarity of illustration.

A first and second heat pump, 1030(1), 1030(2) are examples of heat pump 730, FIG. 7 and heat pump 430, FIG. 4, respectively, and provide heat exchange with ambient air via thermal vents 1028(1), 1028(2). Thermal vents 1028(1), 1028(2) are examples of thermal vents 228(1), 228(2) of FIG. 2 and may include screens or louvers. Located within heat pumps 1030(1), 1030(2) are thermoelectric modules, similar to thermoelectric modules 734(1), 734(2) of FIG. 7, for transferring four individual temperatures via four thermal conductors 1032(1-4). Thermoelectric modules may heat or cool depending on a direction and amount of applied current delivered by controller 1050.

First, second, and third thermal conductors 1032(1), 1032(2), 1032(3) make thermal contact with first, second, and third thermal zones 1070(1), 1070(2), 1070(3), which distribute temperatures established by thermoelectric modules across leg compartment 1010. Thermal zones 1070(1-3) are for example made of conductive material or include a liquid heat exchanger with a fluid loop such as liquid heat exchanger 570, FIG. 5. Temperature sensing devices 1040(1), 1040(2), 1040(3) are examples of temperature sensing device 140, FIG. 1 that provide controller 1050 temperature information for controlling temperature in thermal zones 1070(1-3). System 1000 is used to produce a gradient lengthwise along a patient's legs by controlling thermal conductors 1032(1-4) to desired temperature set points via thermoelectric modules. For example, fourth thermal conductor 1032(4) is controlled to a coldest temperature and first thermal conductor 1032(1) is controlled to a warmest temperature, while second and third thermal conductors 1032(2), 1032(3) are set to appropriate intermediate temperatures.

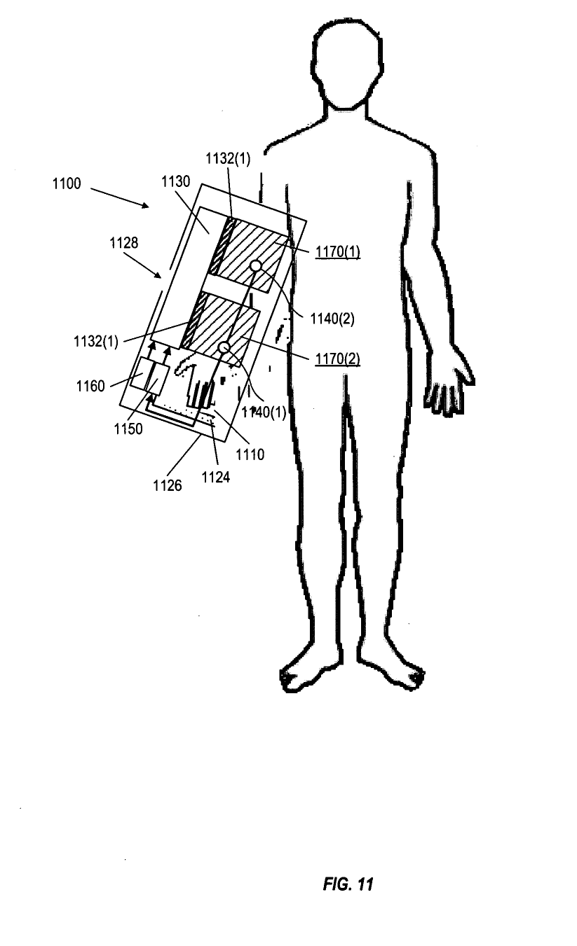

FIG. 11 shows an exemplary temperature gradient system 1100 configured for providing temperature control of an arm. System 1100 is an example of system 1000, FIG. 10 with two thermal zones 1170(1), 1170(2) for controlling arm temperature, which are examples of thermal zones 1070(1-3), FIG. 10. A heat pump 1130 adds or removes heat via ambient air from thermal contacts 1132(1), 1132(2) that are thermally connected to thermal zones 1170(1), 1170(2). Temperature sensing devices 1140(1), 1140(2) provide temperature information to controller 1150 which controls heat pump 1130 and thermoelectric modules located therein (see FIG. 7). Heat pump 1130 is an example of heat pump 730, FIG. 7. An outer cover 1126 has a thermal vent 1128, similar to outer cover 1026 with thermal vents 1028(1), 1028(2) of FIG. 10. An insulating layer 1124 insulates an arm compartment 1110, similar to leg compartment 1010 and insulating layer 1024, FIG. 10. Power converter 1160, which is an example of power converter 1060 of FIG. 10, provides power to heat pump 1130 and if necessary, temperature sensing devices 1140(1), 1140(2). System 1100 is configurable for either a left arm or a right arm and may be used in conjunction with system 1000, FIG. 10 for controlling limb temperature including temperature gradients lengthwise along a patient's limbs.

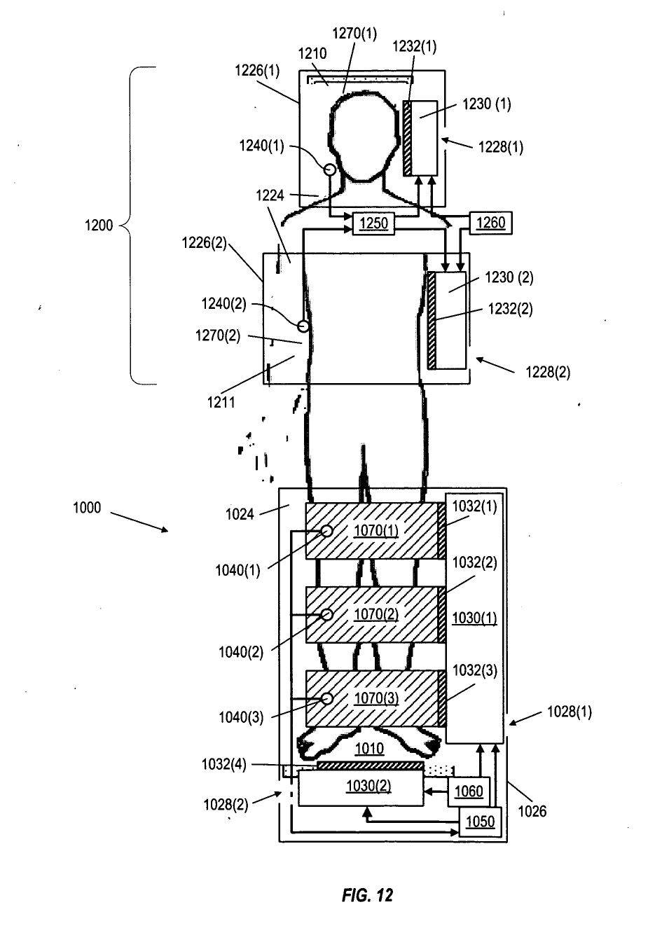

FIG. 12 shows one embodiment of a temperature gradient system 1200 configured for controlling temperature of a head and a torso. System 1200 is an example of system 1000, FIG. 10 and system 1100, FIG. 11 with two thermal zones 1270(1), 1270(2) for controlling head and torso temperature, respectively. System 1200 may be used in conjunction with system 1100 and/or system 1000, as depicted in FIG. 12, for controlling core body temperature separately from limb temperatures. Heat pumps 1230(1), 1230(2) add or remove heat via ambient air from thermal contacts 1232(1), 1232(2) that are thermally connected to thermal zones 1270(1), 1270(2). Temperature sensing devices 1240(1), 1240(2) provide temperature information to controller 1250 which controls heat pump 1230 and thermoelectric modules located therein (see FIG. 4). Heat pumps 1230(1), 1230(2) are examples of heat pump 430, FIG. 4. In an embodiment, a single heat pump is used in place of heat pumps 1230(1), 1230(2), such as heat pump 730, FIG. 7. An outer cover 1226 has two thermal vents 1228(1), 1228(2), similar to outer cover 1026 with thermal vents 1028(1), 1028(2) of FIG. 10. An insulating layer 1224 insulates a head compartment 1210 and a torso compartment 1211 within a first outer cover 1226(1) and a second outer cover 1226(2), respectivley. Power converter 1260, which is an example of power converter 1060 of FIG. 10, provides power to heat pumps 1230(1), 1230(2) and if necessary, temperature sensing devices 1240(1), 1240(2). As depicted in in FIG. 12, controller 1250 and power converter 1260 are located outside outer covers 1226(1), 1226(2), but may be located inside one of outer covers 1226(1), 1226(2) without departing from the scope hereof.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A temperature gradient system, comprising:
an insulated container configured to hold one or more samples;
a first heat pump located at a first end of the insulated container;
a second heat pump located at a second end of the insulated container, wherein the second end is opposite the first end; and
a controller for controlling the first heat pump to maintain a first temperature and the second heat pump to maintain a second temperature, wherein the first and second temperatures are different, thereby maintaining a temperature gradient within the one or more samples.

2. The system of claim 1, the insulated container comprising an inner low emissivity layer, a middle fiberglass layer surrounding the inner layer, and an outer cover surrounding the middle layer, thereby opposing heat transfer to substantially maintain a temperature gradient.

3. The system of claim 2, the outer cover comprising structural support for transporting the one or more samples without damage.

4. The system of claim 2, the outer cover comprising a substantially sealed enclosure thereby preventing external airflow for maintaining a heat transfer barrier.

5. The system of claim 1, the heat pumps comprising a thermal conductor for thermally contacting an end of the one or more samples.

6. The system of claim 1, the one or more samples comprising one or more ice cores.

7. The system of claim 1, the heat pumps comprising at least one thermoelectric module.

8. The system of claim 7, the at least one thermoelectric module comprising a series of cascaded thermoelectric modules.

9. The system of claim 1, further comprising a first temperature sensing device to measure the first temperature and a second temperature sensing device to measure the second temperature.

10. The system of claim 9, the first and second temperature sensing devices being electronically coupled to the controller, thereby providing temperature information to the controller for controlling temperature by adjusting electrical current to the first and second heat pumps.

11. The system of claim 10, comprising a plurality of temperature sensing devices located along the length of the insulated container for determining a temperature gradient.

12. The system of claim 1, the heat pump comprising a heat sink with a plurality of thermally conductive fins.

13. The system of claim 12, the heat pump comprising at least one fan for moving air across the heat sink to outside the insulated container for removing heat.

14. The system of claim 1, the controller comprising;
a microcontroller configured to receive a first and second user defined temperature set point and temperature measurements from first and second temperature sensing devices located near first and second ends of insulated container, respectively;
a memory containing machine readable code configured to determine an amount of electrical current to provide to first and second heat pumps for minimizing a difference between the first and second user defined temperature set points and the first and second temperature measurements, respectively; and
electronic circuitry to provide electrical current to first and second heat pumps for controlling temperature, wherein first and second temperatures are different for maintaining a temperature gradient between first and second ends of the insulated container.

15. A temperature gradient method, comprising:
wrapping a sample with a low emissivity insulating layer;
placing the wrapped sample inside an insulated container;
measuring temperature inside a first end and a second end of the insulated container;
controlling a first heat pump located at a first end of the insulated container to provide a first temperature;
controlling a second heat pump located at a second end of the insulated container, to provide a second temperature, wherein the second temperature is different from the first temperature, thereby maintaining a temperature gradient; and
removing heat from first and second ends with a first heat pump and a second heat pump, respectively.

16. The method of claim 15, wherein wrapping the sample comprises wrapping an ice core.

17. The method of claim 15, comprising measuring temperature at a plurality of locations along the length of the sample and determining temperature gradient.

18. The method of claim 15, the step of removing heat comprising moving air with a fan across a heat sink containing a plurality of thermally conductive fins and through vents that pass through the insulated container to outside.

19. A temperature gradient system, comprising:
an insulated container having a compartment configured to accommodate a portion of a human body;
a plurality of thermal zones for controlling temperature of the compartment;
at least one heat pump thermally connected to the plurality of thermal zones, wherein the at least one heat pump includes one or more thermoelectric modules; and
a controller for controlling the one or more thermoelectric modules to maintain a desired temperature in the thermal zones for controlling temperature to the portion of the human body.

20. The system of claim 19, wherein the at least one heat pump includes a heat exchanger to add or remove heat from the one or more thermoelectric modules.

21. The system of claim 19, further comprising a plurality of temperature sensing devices to measure temperatures of the plurality of thermal zones, the temperature sensing devices being electronically coupled to the controller, thereby providing temperature information to the controller for controlling temperature by adjusting electrical current to the one or more thermoelectric modules.

22. The system of claim 21, the temperatures of the plurality of thermal zones forming a temperature gradient within the compartment.

23. The system of claim 22, the compartment comprising a leg compartment configured to accommodate legs for controlling temperature lengthwise along human legs.

24. The system of claim 22, the compartment comprising an arm compartment configured to accommodate arms for controlling temperature lengthwise along human arms.

25. The system of claim 22, the compartment comprising a head compartment and a torso compartment configured to accommodate a human head and torso, respectively, for controlling a head temperature and a torso temperature.
